(19) United States
(12) Patent Application Publication
(10) Pub. No.: US 2024/0189548 A1
(43) Pub. Date: Jun. 13, 2024

(54) APPARATUS AND METHODS FOR TRANSSEPTAL CATHETERIZATION

(71) Applicant: St. Jude Medical, Cardiology Division, Inc., St. Paul, MN (US)

(72) Inventors: Troy Tegg, Elk River, MN (US); Hong Cao, Maple Grove, MN (US); Salo Arias, Brooklyn Park, MN (US)

(21) Appl. No.: 18/537,213

(22) Filed: Dec. 12, 2023

Related U.S. Application Data
(60) Provisional application No. 63/387,158, filed on Dec. 13, 2022.
(60) Provisional application No. 63/582,012, filed on Sep. 12, 2023.

(51) Int. Cl. A61M 25/01; A61M 25/00; A61M 25/09; A61M 29/02

(52) U.S. Cl. CPC A61M 25/0147; A61M 25/0026; A61M 25/09; A61M 29/02

(57) ABSTRACT

An apparatus for transseptal catheterization includes a dilator with an energy delivery element attached to its distal end. The energy delivery element is configured to deliver sufficient energy to a tissue, such as the fossa ovalis, adjacent the distal end to permit the dilator to penetrate the tissue and cross into the left atrium. The energy delivery element can be a radiofrequency electrode, a pulsed field ablation electrode, an ultrasound transducer, or the like. A guidewire and/or introducer may also be included to facilitate the transseptal catheterization.

### CROSS-REFERENCE TO RELATED APPLICATIONS

The present application claims the benefit of U.S. provisional application No. 63/387,158, filed 13 Dec. 2022 (“the '158 provisional”) and U.S. provisional application No. 63/582,012, filed 12 Sep. 2023 (“the '012 provisional”). The '158 and '012 provisionals are hereby incorporated by reference as though fully set forth herein.

### BACKGROUND

The present disclosure relates generally to apparatus and methods for penetrating tissue within the body. In particular, the instant disclosure relates to apparatus and methods for crossing the interatrial septum from the right atrium in order to access the left atrium, a procedure known and referred to herein as “transseptal catheterization.”

The human heart includes a right ventricle, a right atrium, a left ventricle, and a left atrium. The right atrium is in fluid communication with the superior vena cava (SVC) and the inferior vena cava (IVC). The tricuspid valve separates the right atrium from the right ventricle. The right atrium is separated from the left atrium by a septum that includes a thin membrane known as the fossa ovalis.

There are several diagnostic and therapeutic procedures in which a catheter is advanced within a guide (or introducer) sheath or over a guidewire, through a subject's vasculature, and into various chambers and across valves of the heart. Certain such procedures require the catheter to be placed in the subject's left atrium.

Yet, the left atrium is the most difficult chamber of the heart to access transluminally. Access to the left atrium through the pulmonary artery is not possible, and approaches from the left ventricle are challenging.

Accordingly, one accepted method of accessing the left atrium is transseptal catheterization. In transseptal catheterization, a catheter is inserted through the femoral or left subclavian vein into the right atrium, followed by penetration of the interatrial septum, typically across the fossa ovalis, to gain entry to the left atrium.

In many cases, transseptal catheterization requires the use of multiple devices, which may be separate components or part of an integrated kit. These devices generally include an introducer sheath, a dilator, and a needle. In some instances, a guidewire is also provided over which the introducer may be inserted into the body. Similarly, the needle may include a stylet in order to enhance its pushability.

One conventional approach to transseptal catheterization, which will be familiar to those of ordinary skill in the art, involves advancing the dilator through the patient's vasculature, optionally with the aid of a guide wire and/or introducer sheath, to the fossa ovalis. Once the fossa ovalis is located, the needle can be advanced through the dilator to penetrate the interatrial septum. Next, the dilator (and/or introducer sheath, as applicable) is advanced through the resulting perforation and into the left atrium. The needle can then be removed and exchanged for a guidewire into the left atrium before further advancing the dilator and introducer sheath. Afterwards, the dilator and guidewire can be removed altogether and a diagnostic or therapeutic medical device, such as an electrophysiology mapping catheter or an ablation catheter, can be inserted in its place and advanced into the left atrium.

Alternatively, the introducer sheath and dilator can be removed with the guidewire remaining in the left atrium. Subsequently, a new device can be advanced over the guidewire and into the left atrium.

### BRIEF SUMMARY

Disclosed herein is an apparatus for transseptal catheterization, including: a dilator having a proximal end, a distal end, and a lumen extending between the proximal end and the distal end; and an energy delivery element attached to the distal end of the dilator and configured to deliver sufficient energy to a tissue adjacent the distal end of the dilator to permit the dilator to penetrate the tissue.

In embodiments of the disclosure, the energy delivery element includes an electrode, such as a radiofrequency ablation electrode or a pulsed field ablation electrode.

The energy delivery element can include a hollow cylindrical element inserted into the lumen at the distal end of the dilator. Alternatively, the energy delivery element can include a solid cylindrical element inserted into the lumen at the distal end of the dilator.

In embodiments where the energy delivery element is a hollow cylindrical element, an interior-facing energy-emitting surface of the energy delivery element can be covered with an energy-inhibiting material, leaving at least a portion of the distal-facing energy-emitting surface of the energy delivery element exposed to contact tissue. For instance, it is contemplated that between one-quarter and three-quarters of the distal-facing energy-emitting surface may be exposed to contact tissue, such that the energy delivery element creates a slit, rather than a hole, in the fossa ovalis.

A temperature sensor, such as a thermocouple or thermistor, can be carried by the dilator proximate the distal end. A magnetic localization element can also be carried by the dilator proximate the distal end.

According to aspects of the disclosure, the apparatus further includes a guidewire configured to be inserted through the lumen of the dilator. The lumen of the dilator can include both a central lumen terminating at an opening in the distal end of the dilator and a side lumen terminating at a side port through a wall of the dilator proximate the distal end of the dilator. It is contemplated that the guidewire may be configured for insertion through either the main lumen or the side lumen.

In yet other aspects of the disclosure, the apparatus further includes an introducer sheath having a distal end, a proximal end, and a lumen extending therebetween. The dilator is configured to be inserted through the lumen of the introducer sheath.

The dilator can also include a hypotube, and the hypotube can define the lumen of the dilator. In some of the embodiments disclosed herein, the hypotube is made of an electrically-conductive material, such as stainless steel, and can be conductively coupled to the energy delivery element to supply power thereto. Optionally, the hypotube can include a plurality of slots cut into its wall, with each slot extending at least partially around the perimeter of the hypotube and at least partially through the wall of the hypotube.

In still other embodiments, the hypotube can include a spring coil.

The dilator lumen can include a main (e.g., central) lumen and at least one secondary lumen. In some embodiments of the disclosure, the secondary lumen is a conductor lumen that accommodates an electrical conductor that is conductively coupled to the energy delivery element to supply power thereto. In other embodiments of the disclosure, the secondary lumen is a pressure-measuring lumen having an open distal end, thereby facilitating pressure measurement at the distal end of the dilator.

The instant disclosure also provides an apparatus for transseptal catheterization, including: a dilator having a proximal end, a distal end, and a lumen extending therebetween; and a guidewire configured to be inserted through the lumen of the dilator and having a distal end, wherein at least one of the distal end of the dilator and the distal end of the guidewire comprises an energy delivery element configured to deliver sufficient energy to an adjacent tissue to permit penetration of the adjacent tissue.

According to aspects of the disclosure, the energy delivery element includes an electrode attached to the distal end of the dilator. In other aspects of the disclosure, the energy delivery element includes an exposed conductive distal end of the guidewire.

It is also contemplated that the apparatus can include an introducer sheath having a distal end, a proximal end, and a lumen extending therebetween. The dilator can be configured to be inserted through the lumen of the introducer sheath.

Also disclosed herein is a method of performing a transseptal catheterization, including the steps of: advancing a medical device through a vasculature and into a right atrium of a heart, wherein the medical device has a proximal end and a distal end and further includes a lumen extending from the proximal end to the distal end and an energy delivery element secured to the distal end; bringing the energy delivery element of the medical device into contact with a penetration site on an interatrial septum separating the right atrium from a left atrium of the heart; causing energy sufficient to penetrate the interatrial septum to be applied to the penetration site through the energy delivery element; and advancing the medical device through a penetration in the interatrial septum and into the left atrium of the heart.

It is contemplated that the medical device includes a dilator.

The energy delivery element can include an electrode, and the step of causing energy sufficient to penetrate the interatrial septum to be applied to the penetration site through the energy delivery element can include causing radiofrequency energy sufficient to penetrate the interatrial septum to be applied to the penetration site through the electrode.

The method can also include advancing a guidewire through the lumen of the medical device after advancing the medical device through the penetration in the interatrial septum and into the left atrium of the heart. For instance, the lumen can include a main lumen and a side lumen, with the side lumen terminating at a side port through a wall of the medical device, and the guidewire can be advanced through the side lumen and out of the side port.

Still further, the instant disclosure provides a dilator including: an elongate body having a proximal end and a distal end and defining at least one lumen; an energy delivery element attached to the distal end of the elongate body, the energy delivery element including a distal-facing surface, wherein a portion of the distal-facing surface of the energy delivery element is covered by an energy-inhibiting material; and an electrical conductor extending through the elongate body and conductively coupled to the energy delivery element. According to aspects of the disclosure, the electrical conductor can be an electrically-conductive hypotube that in turn defines the at least one lumen.

In yet additional aspects, the instant disclosure provides an apparatus for transseptal catheterization, including: an introducer including an introducer hub attached to a proximal end of the introducer; a dilator including an elongate body and an energy delivery element attached to a distal end of the elongate body, wherein the energy delivery element is configured to deliver sufficient energy to a tissue adjacent the distal end of the dilator to permit the dilator to penetrate the tissue; and an adapter having a distal end, a proximal end, and a lumen extending between the distal end of the adapter and the proximal end of the adapter, wherein the lumen is configured to receive the elongate body of the dilator therethrough. The adapter further includes: a connector at the distal end of the adapter, wherein the connector is configured to releasably secure the adapter to the introducer hub; and a fixation device, such as a screw, clamp, or valve (e.g., a hemostasis valve), at the proximal end of the adapter, wherein the fixation device is configured to releasably secure the adapter to the dilator when the elongate body of the dilator is received within the lumen of the adapter.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates representative elements of an apparatus for transseptal catheterization according to aspects of the present disclosure.

FIG. 2 illustrates a dilator for transseptal catheterization according to embodiments disclosed herein.

FIG. 3 is a close-up and cut-away view of the proximal end of the dilator of FIG. 2.

FIG. 4A is a close-up and cut-away view of the distal end of the dilator of FIG. 2 according to various embodiments of the disclosure.

FIG. 4B is a perspective view of the distal end of the dilator of FIG. 2 and illustrates an embodiment of the disclosure where the energy delivery element is partially covered by an energy-inhibiting material.

FIG. 4C illustrates an embodiment of an energy delivery element having a portion of a circumferential wall cut-away to form an arcuate energy emitting surface.

FIG. 5 is a close-up and cut-away view of the distal end of a dilator according to another embodiment of the instant disclosure including a generally solid energy delivery element.

FIG. 6 is a close-up and cut-away view of the distal end of a dilator illustrating additional aspects of the instant disclosure.

FIG. 7 is a close-up and cut-away view of the distal end of a dilator illustrating still further aspects of the instant disclosure.

FIG. 8 illustrates the use of a guidewire with the dilator of FIG. 2.

FIG. 9 is a diagrammatic and block diagram view of an illustrative system such as may be used in accordance with aspects of the instant disclosure.

FIGS. 10-12 illustrate various steps in a transseptal catheterization procedure.

FIG. 13 illustrates an adapter to secure a dilator to an introducer according to aspects of the instant disclosure.

FIG. 14A is a cross-sectional view of the adapter of FIG. 13.

FIG. 14B is an exploded view of the adapter of FIG. 13.

FIG. 15 illustrates use of the adapter of FIG. 13 to secure a dilator to an introducer.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

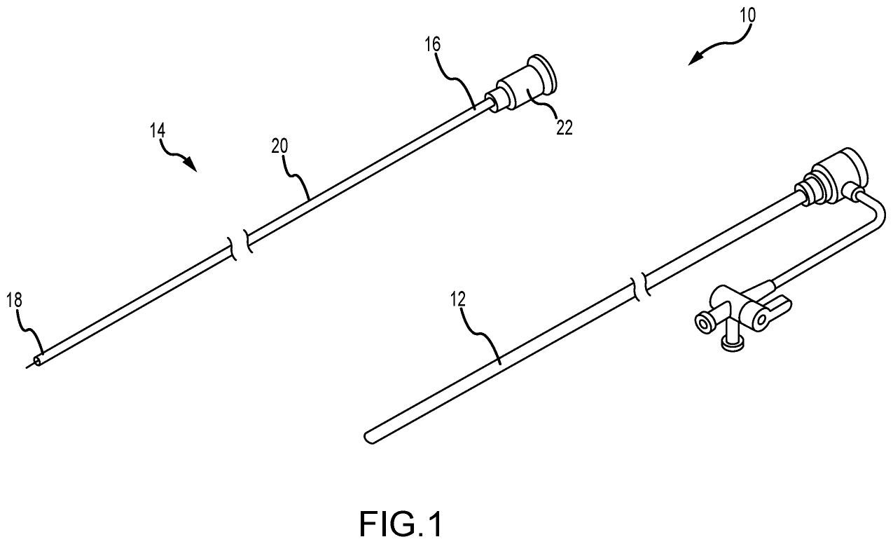

Referring now to the drawings, FIG. 1 illustrates various elements of a representative apparatus 10 for transseptal catheterization, including an introducer 12 and a dilator 14. Insofar as the construction of introducer 12 will be familiar to those of ordinary skill in the art, it will not be discussed in further detail herein. By way of example only, however, introducer 12 may be the Agilis™ NXT Steerable Introducer (Abbott Laboratories; Abbott Park, Illinois).

Many aspects of the structure of dilator 14 will also be familiar to those of ordinary skill in the art. Accordingly, dilator 14 will be described herein only to the extent necessary to understand the instant disclosure.

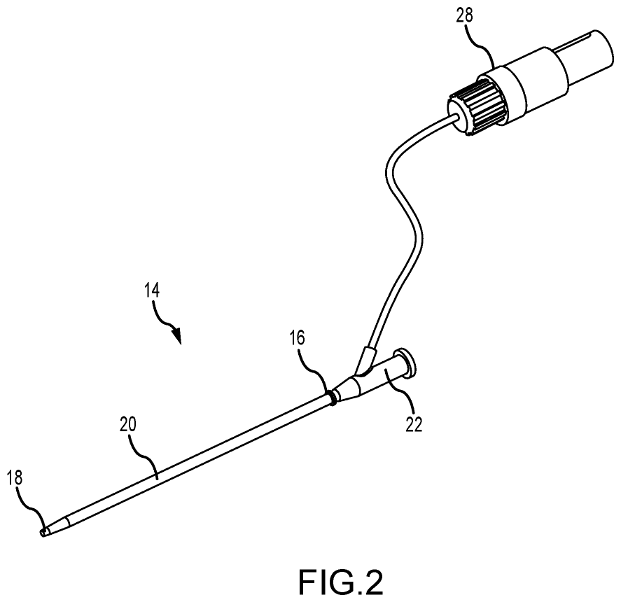

As shown in FIGS. 1 and 2, dilator 14 generally includes a proximal end 16, a distal end 18, and a body 20 extending therebetween. Dilator 14 can be provided in various outer diameters (i.e., French size), in various lengths, and/or with various preset or adjustable curvatures of distal end 18, as may be desirable for any particular application of dilator 14. Accordingly, the specific dimensions and shapes of the embodiments of dilator 14 depicted and described herein are intended to be exemplary and illustrative rather than limiting. Indeed, those of ordinary skill in the art will appreciate that various modifications can be made thereto without departing from the scope of the instant disclosure. For instance, although in certain embodiments of the disclosure, the outer diameter of body 20 may be about 0.110 inches (sized to fit, for example, in a standard 8.5 French introducer 12), the outer diameter may be made larger or smaller depending on the intended use of dilator 14 and/or the French size of introducer 12 to be used in conjunction therewith.

Likewise, various materials suitable for use in the construction of body 20 of dilator 14 will be known to those of ordinary skill in the art. By way of illustration only, however, such materials include, without limitation, various nylon polymers (e.g., Nylon 12), polyether block amide elastomers in various durometers (e.g., PEBAX 72D (Arkema Inc.; King of Prussia, PA), high-density polyethylene (HDPE), and other thermoplastics.

It is further contemplated that body 20 may combine sections of different materials. These sections may be arranged in layers, such as by co-extrusion, and/or in abutment along the length of body 20. As but one example, in certain embodiments of the disclosure, the majority of body 20 may be made of Nylon 12, while about a two-inch long segment of body 20 proximate distal end 18 may be made of PEBAX 72D. This configuration is advantageous insofar as Nylon 12 is stiffer and can improve pushability of the proximal portion of body 20, while PEBAX 72D is softer and renders the distal end of body 20 more compliant to pass through and/or be deflected by an introducer or sheath.

A dilator hub 22, which will be familiar to the ordinarily-skilled artisan, may be attached to proximal end 16 of dilator 14. Amongst other functions, dilator hub 22 can be used to deliver irrigation to distal end 18 of dilator 14 and/or for aspiration of the device to avoid introduction of air into the bloodstream. Further, a hemostasis valve adapter including a Luer lock fitting, can be attached to dilator hub 22 with guidewire 35 (described further below) inserted into dilator 14.

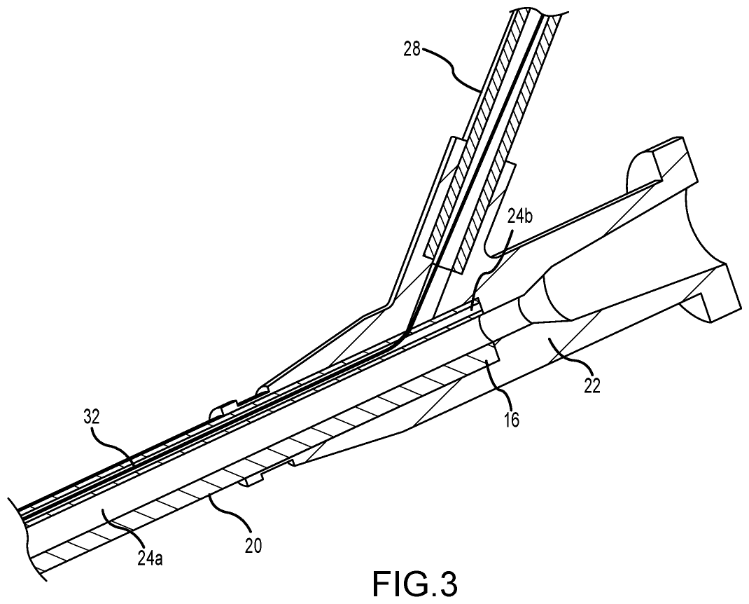

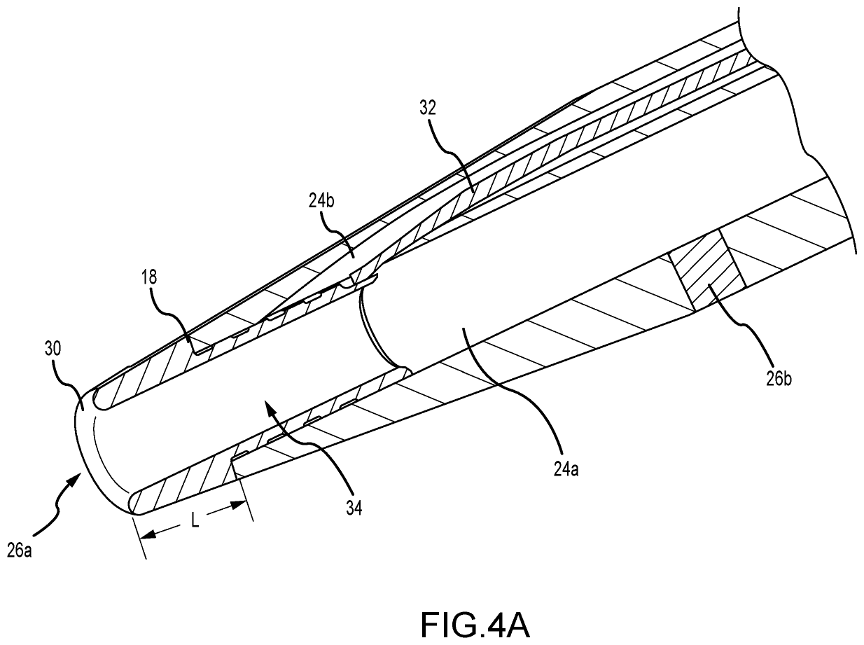

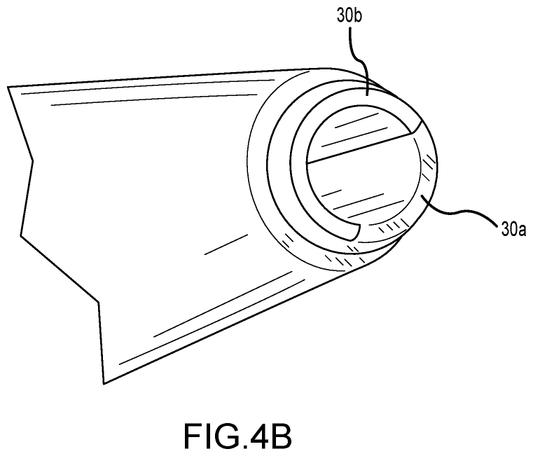

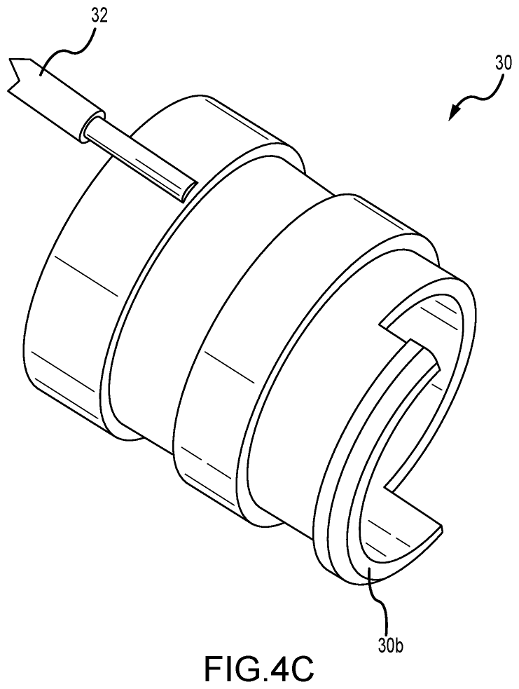

Body 20 defines a lumen 24, visible in FIG. 3, which is a close-up, cross-sectional view of proximal end 16 of dilator 14, and in FIG. 4A, which is a close-up, cross-sectional view of distal end 18 of dilator 14. In the embodiment illustrated in FIGS. 3 and 4, lumen 24 includes a main lumen 24a having a diameter of about 0.050 inches and a side lumen 24b having a diameter of about 0.019 inches. It should be understood, however, that the representation of body 20 in FIGS. 3 and 4 is merely illustrative and that the number, size, shape, and/or arrangement of lumen(s) 24 can be tailored as desired in connection with any particular application of dilator 14.

In the embodiment of the disclosure shown in FIGS. 3 and 4, main lumen 24a opens proximally through dilator hub 22 and opens distally, either through a distally-facing opening 26a or a side port 26b in body 20 proximate distal end 18 of dilator 14. Side lumen 24b opens proximally, either through dilator hub 22 or through an opening in connector 28 (discussed in greater detail below). Side lumen 24b may have a closed distal end as shown in FIG. 4A, an arrangement that may be particularly desirable where side lumen 24b is used to route an electrical conductor to supply ablative energy to an energy delivery element as described below. It is within the scope of the instant disclosure, however, for side lumen 24b to open distally, such as through a distally-facing opening 26a or a side port 26b.

An energy delivery element 30 is attached to distal end 18 of dilator 14. Energy delivery element 30 is operable to deliver sufficient energy to a tissue adjacent distal end 18 of dilator 14 to permit dilator 14 to penetrate that tissue.

For example, in embodiments of the disclosure, energy delivery element 30 is an electrode suitable for delivering radiofrequency (RF) energy to adjacent tissue, such as a platinum-iridium electrode. To this end, energy delivery element 30 may be conductively coupled to a source of RF energy, such as the Abbott Laboratories Ampere™ RF Ablation Generator, or an electrosurgical generator, such as the VIO® 300D electrosurgical system (Erbe USA, Inc.; Marietta, Georgia), via an electrical conductor 32 that conductively couples energy delivery element 30 to connector 28, which, in turn, connects to the energy source. Electrical conductor 32 may be routed through body 20 (e.g., embedded in the wall of body 20) or, alternatively, routed through side lumen 24b.

In other embodiments of the disclosure, energy delivery element 30 may be an electrode configured to deliver irreversible electroporation therapy (IRE) (also known as pulsed field ablation (PFA)). Such an electrode can likewise be conductively coupled to a suitable generator via connector 28.

In still other embodiments of the disclosure, energy delivery element 30 may include one or more acoustic elements (e.g., ultrasound transducers).

In yet further embodiments of the disclosure, energy delivery element can be capable of generating electromagnetic fields strong enough to cause enough damage to adjacent tissue to enable penetration by dilator 14.

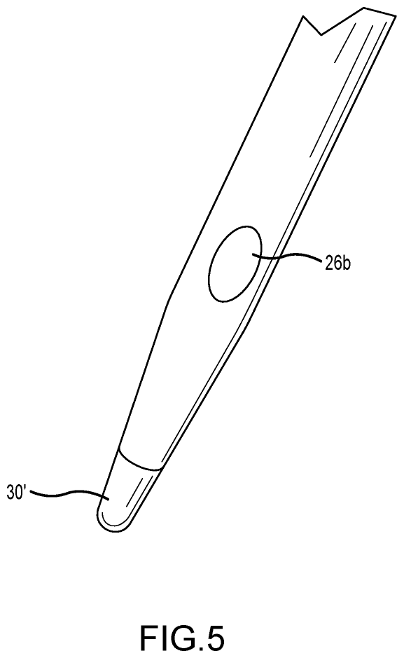

According to certain aspects of the disclosure, energy delivery element 30 is a hollow cylindrical element inserted into main lumen 24a at distal end 18. As used herein, the term “cylindrical” is not limited to right circular cylinders, but rather is intended to encompass cylinders with non-circular bases and/or cylinders that taper or otherwise vary in cross-sectional dimension along their height. Indeed, FIGS. 4A, 4B, and 5 (described below) illustrate that energy delivery element 30 may taper distally, a shape that the ordinarily-skilled artisan will appreciate facilitates transseptal crossings at the fossa ovalis.

FIG. 5 shows an alternative energy delivery element 30′ that can be utilized in other aspects of the disclosure. More particularly, energy delivery element 30′ shown in FIG. 5 is a solid cylindrical element. Thus, energy delivery element 30′ obstructs distally-facing opening 26a (e.g., main lumen 24a has a closed distal end). Side port 26b, however, remains open (and may allow access to side lumen 24b and/or main lumen 24a).

Those of ordinary skill in the art will appreciate the desirability of promoting energy delivery from energy delivery element 30 into the tissue to be penetrated (e.g., the interatrial septum), rather than into the atrial blood pool. Energy delivery element 30 can be shaped and/or treated accordingly. For example, the extent to which energy delivery element 30 protrudes beyond distal end 18 of dilator 14, denoted “L” in FIG. 4, can be relatively short, on the order of at least about 0.015 inches.

As another example, the interior-facing surface 34 of energy delivery element 30 can be treated with an energy-inhibiting material (e.g., an electrical insulator in the case of an RF electrode). Thus, the energy-emitting surface of energy delivery element 30 can be substantially limited to the distally-facing portion of energy delivery element 30 that will be in contact with tissue. Of course, in other embodiments of the disclosure, interior-facing surface 34 can be left untreated, and therefore conductive.

As still another example, the distally-facing energy-emitting surface of energy delivery element 30 need not be fully circular (or another closed-loop shape). Rather, some portion of the distally-facing energy-emitting surface (e.g., between about one-fourth and about three-fourths of the distally-facing energy-emitting surface) can be treated with an energy-inhibiting material (e.g., an electrical insulator in the case of an RF or PFA electrode), such that energy delivery element 30 will form a slit or flap, rather than a hole, in the fossa ovalis. This is shown, for example, in FIG. 4B, where the material of body 20 covers (e.g., by overmolding) about half (30a) of the distally-facing energy-emitting surface of energy delivery element 30, leaving an arcuate exposed portion (30b) capable of delivering energy to tissue.

Further, it is desirable for exposed portion 30b to be on inside edge of the curvature when dilator 14 is curved. This configuration facilitates engagement of exposed portion 30b with tissue (e.g., of the fossa ovalis).

Various approaches for forming the partially-exposed, partially-covered arrangement of FIG. 4B are contemplated. According to some aspects of the disclosure, for example, exposed portion 30b can be masked prior to reflow processing of dilator 14. Once reflow processing is complete (e.g., body 20 has cooled and solidified), the mask can be removed to reveal exposed portion 30b, with the covered portion 30a remaining covered by overmolding.

Alternatively, in other aspects of the disclosure, exposed portion 30b of the desired size may be formed by material removal after reflow processing is complete (e.g., body 20 has cooled and solidified, including over substantially all of energy delivery element 30).

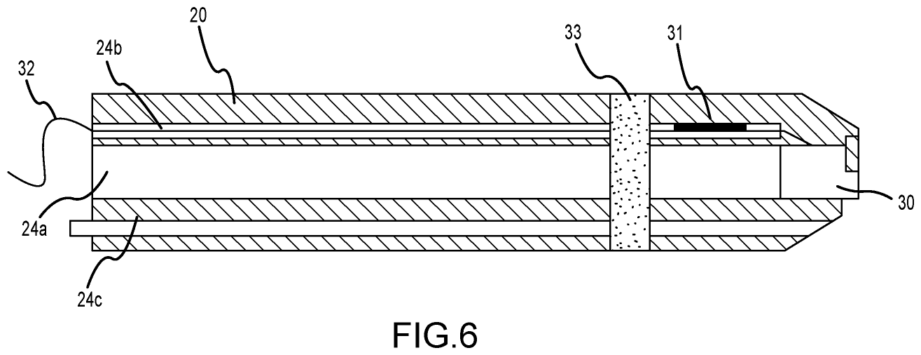

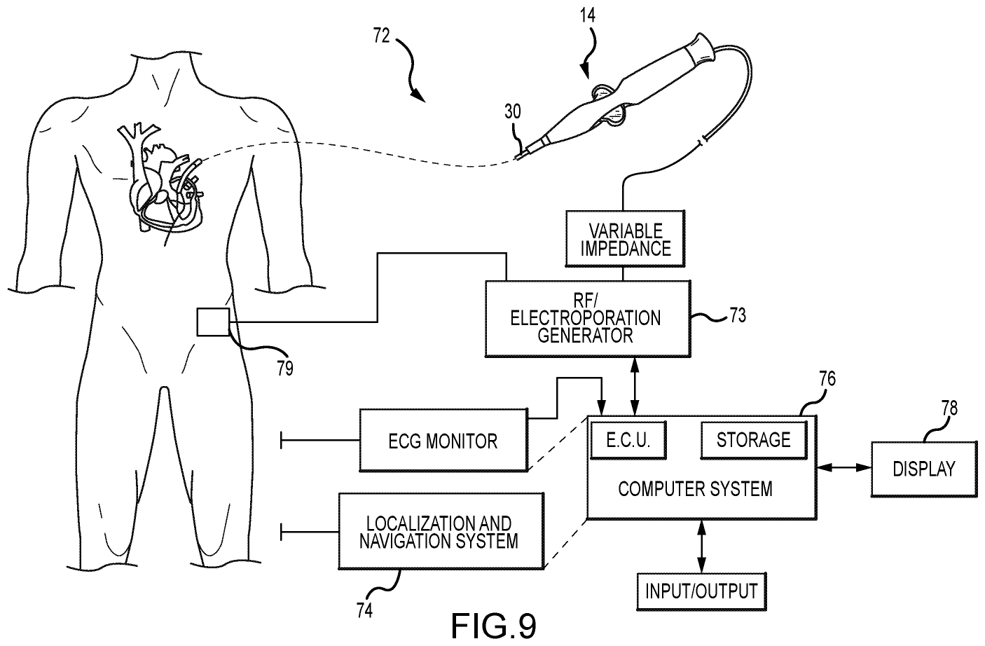

In still other embodiments, such as shown in FIG. 4C, a portion of the circumferential wall of energy delivery element 30 can be cut away (and, optionally, replaced with an insulating material, such as the material of body 20) to form arcuate energy delivery portion 30b.

Where energy delivery element 30 is an electrode, it can also advantageously be localized (and, optionally, visualized) using an impedance-based electroanatomical mapping system 74 (see FIG. 9), such as Abbott Laboratories' EnSite™ X electrophysiology system. Alternatively or additionally, a secondary electrode 33 (such as a ring electrode, a segmented ring electrode, or the like) may be provided on body 20, as shown in FIG. 6, to enhance localization and/or visualization of dilator 14 within electroanatomical mapping system 74. As a further advantage, energy delivery element 30 and secondary electrode 33 can be used as a bipolar pair to map electrophysiological activity within the heart. This, in turn, can enhance a practitioner's ability to locate the fossa ovalis.

Dilator 14 can be equipped with one or more magnetic localization elements 31 (e.g., magnetic field-sensing coils) in order to enable the localization (and, optionally, visualization) of dilator 14 using magnetic field-based modalities, either as an alternative to or in addition to impedance-based modalities that track energy delivery element 30 and/or secondary electrode 33.

FIG. 6 further illustrates an additional side lumen 24c that is open distally. As such, side lumen 24c can be used to measure the pressure at distal end 18 of dilator 14 (e.g., using pressure measuring equipment coupled to the proximal end of side lumen 24c). Such measurements can be used to confirm transseptal crossing by detecting (e.g., in computer system 76 and/or electroanatomical mapping system 74) the pressure change associated with moving from the right atrium into the left atrium.

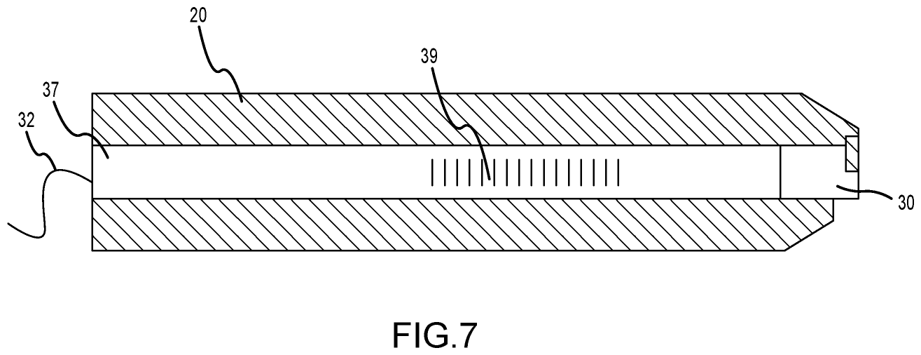

FIG. 7 illustrates distal end 18 of dilator 14 according to still further aspects of the instant disclosure. In the embodiment shown in FIG. 7, a hypotube 37 is embedded within body 20 to increase the stiffness thereof, such as by extruding body 20 over hypotube 37. Thus, main lumen 24a is defined by hypotube 37 rather than by body 20.

Hypotube 37 may have any inner diameter, outer diameter, wall thickness, and length that may be desirable for a given application of dilator 14. By way of example only, the outer diameter of hypotube 37 may be about 0.050 inches; the inner diameter of hypotube 37 may be about 0.045 inches; and the wall of hypotube 37 may be about 0.0025 inches thick.

It is contemplated that hypotube 37 may be made of a conductive material, such as stainless steel (e.g., 304 stainless steel). In such aspects of the disclosure, electrical conductor 32 can be attached to the proximal end of hypotube 37 and energy delivery element 30 can be attached to the distal end of hypotube 37, such that hypotube 37 can be used in place of electrical conductor 32. It should be understood, however, that hypotube 37 need not be conductively coupled to energy delivery element 30; in other words, electrical conductor 32 and hypotube 37 are both present in certain aspects of the disclosure.

The inner surface of hypotube 37 (that is, the surface of hypotube 37 facing main lumen 24a) may be insulated, such as by use of a polytetrafluoroethylene (PTFE) or polyimide (PI) liner or coating, to isolate electrical current flowing through hypotube 37 from any devices that may be advanced through main lumen 24a (such as guidewire 35 described below).

Use of a stainless steel hypotube 37 also allows a practitioner to plastically deform distal end 18 of dilator 14 into various shapes that may be useful in a particular procedure. To further enhance this formability, some or all of hypotube 37 may include slots 39 and/or a coil (e.g., a spring coil). Slots 39 are oriented substantially perpendicular to the longitudinal axis of hypotube 37 and may extend any amount around the perimeter of hypotube 37 and may extended either entirely or partially through the wall of hypotube 37. By way of illustration only, however, in some embodiments of the disclosure, each slot has a length of about 0.2 mm and the slots are spaced about 1 mm apart from each other.

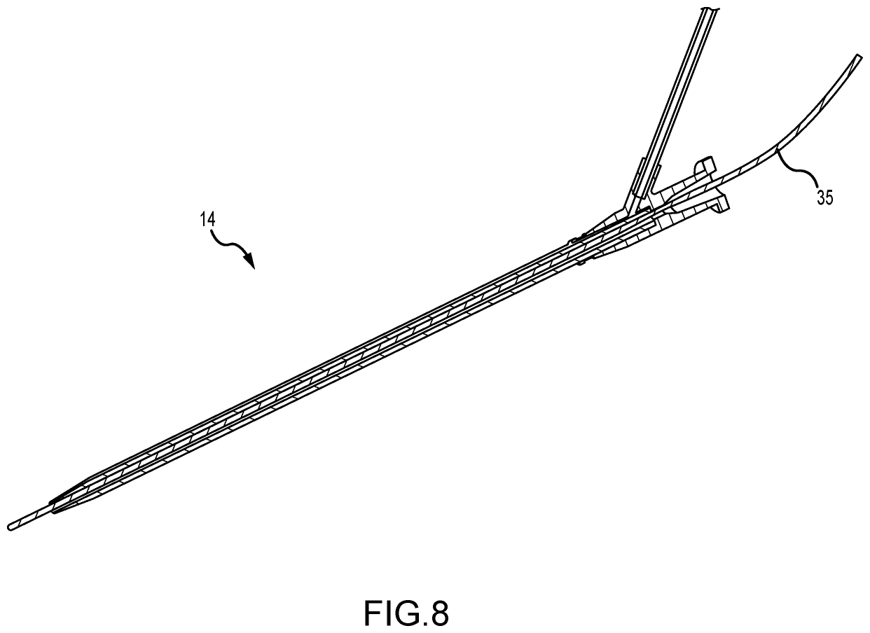

Apparatus 10 can also include a guidewire 35, shown in FIG. 8. Guidewire 35 is configured to be inserted through hub 22 and lumen 24 during a transseptal catheterization, as described below.

FIG. 9 is a diagrammatic and block diagram view of a system 70 that may be utilized in accordance with aspects of the instant disclosure. FIG. 9 schematically illustrates dilator 14 as connected to electronics 72 within system 70. As those of ordinary skill in the art will appreciate, and as shown in FIG. 9, electronics 72 may include an energy generator 73, an electroanatomical mapping system 74, a computer system 76, a display 78, and the like. Insofar as the components of system 70 will be familiar to those of ordinary skill in the art, they need not be described in detail herein.

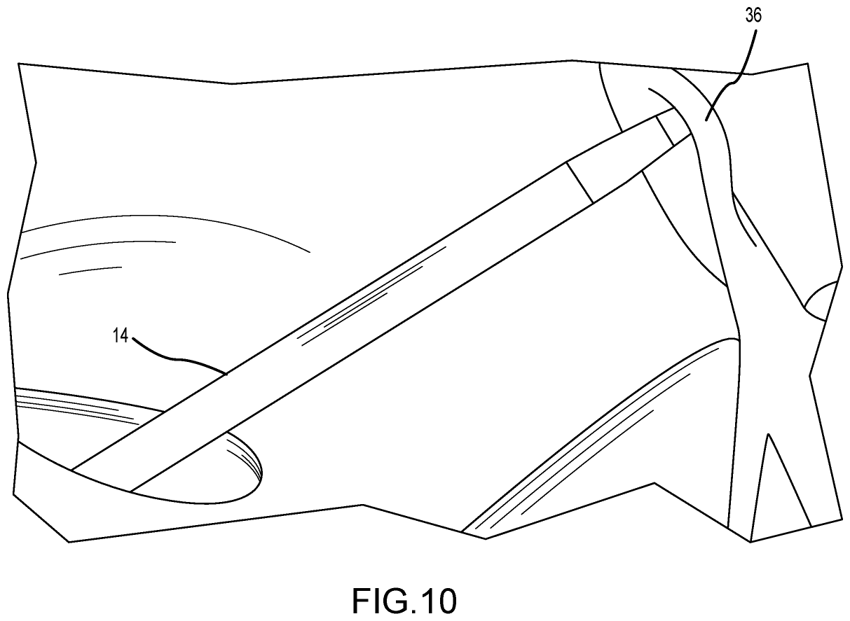

In use, and as illustrated in FIG. 10, dilator 14 is advanced through a subject's vasculature, optionally with the aid of introducer 12 and/or guidewire 35, and into the subject's right atrium. Distal end 18 of dilator 14 is used to locate the fossa ovalis 36 in a manner known to those of ordinary skill in the art (though, as noted above, energy delivery element 30 and/or secondary electrode 33 may simplify this aspect of the procedure insofar as, unlike extant transseptal catheterization apparatus, it allows dilator 14 to be localized and visualized via an electroanatomical mapping system and/or enables bipolar electrophysiological measurements that may enhance a practitioner's ability to locate the fossa ovalis).

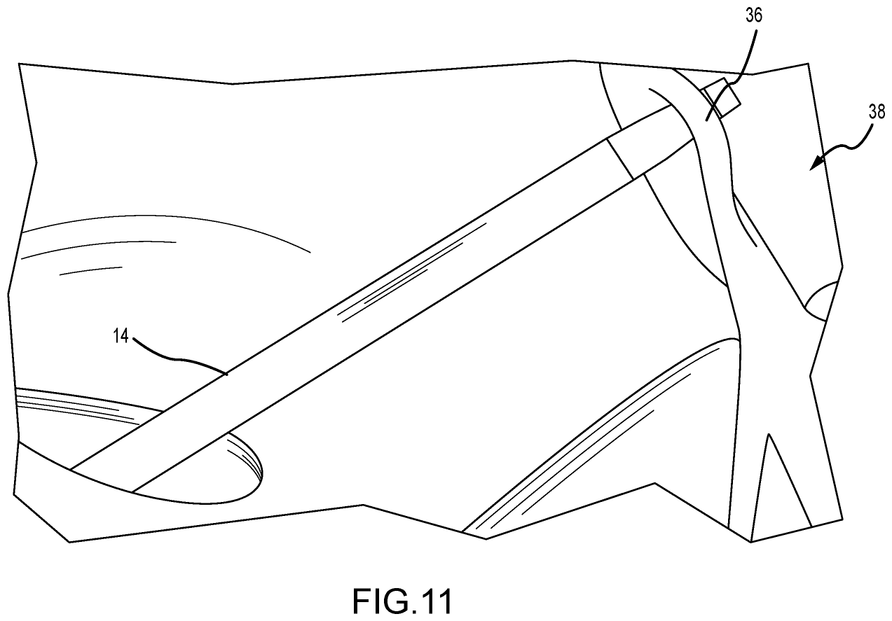

Once tenting of fossa ovalis 36 is observed, energy delivery element 30 (e.g., via energy generator 73) is activated to deliver sufficient energy to allow dilator 14 (and, optionally, introducer 12) to penetrate fossa ovalis 36 and cross into the left atrium 38 as shown in FIG. 11 (referred to herein as the “penetrating energy”). During energy delivery, guidewire 35 may be retracted into dilator 14, away from energy delivery element 30 and out of the energy delivery path.

As briefly mentioned above, various modalities for delivery of the penetrating energy are contemplated. For example, in some embodiments of the disclosure, energy delivery element 30 is a radiofrequency (RF) ablation electrode, and energy generator 73 delivers sufficient RF energy to penetrate fossa ovalis 36 and cross into the left atrium 38.

In other embodiments of the disclosure, energy delivery element 30 is configured to deliver irreversible electroporation therapy (IRE) (also known as pulsed field ablation (PFA) therapy), and energy generator 73 delivers the energy pulses required to penetrate fossa ovalis 36 and cross into left atrium 38. Where only a single energy delivery element 30 is present on dilator 14, dilator 14 can serve as a monopolar IRE/PFA probe. Alternatively, if dilator 14 includes two or more energy delivery elements (e.g., energy delivery element 30 and secondary electrode 33), dilator 14 can be used to as a bipolar IRE/PFA or RF probe.

In still further embodiments of the disclosure, energy delivery element 30 may include one or more acoustic elements (e.g., ultrasound transducers), and energy generator 73 is an acoustic generator.

In yet further embodiments of the disclosure, energy generator 73 powers energy delivery element 30 to generate an electromagnetic field strong enough to cause enough damage to fossa ovalis 36 to enable penetration by dilator 14.

In some embodiments of the disclosure, the penetrating energy can be about 10 Watts of energy applied over a time interval of about 5 seconds. In other embodiments of the disclosure, the penetrating energy can be about 429V peak 30 W maximum applied over a time interval of about 2 seconds and in multiple bursts. It should be understood, however, that other power levels and/or time intervals are regarded as within the spirit and scope of the instant disclosure.

In some embodiments of the disclosure, energy delivery element 30 operates as a source electrode for the penetrating energy and a patch electrode 79 operates as the sink for the penetrating energy. In other embodiments of the disclosure, the penetrating energy originates at energy delivery element 30, passes through guidewire 35, and sinks through patch electrode 79. Still other paths are also contemplated.

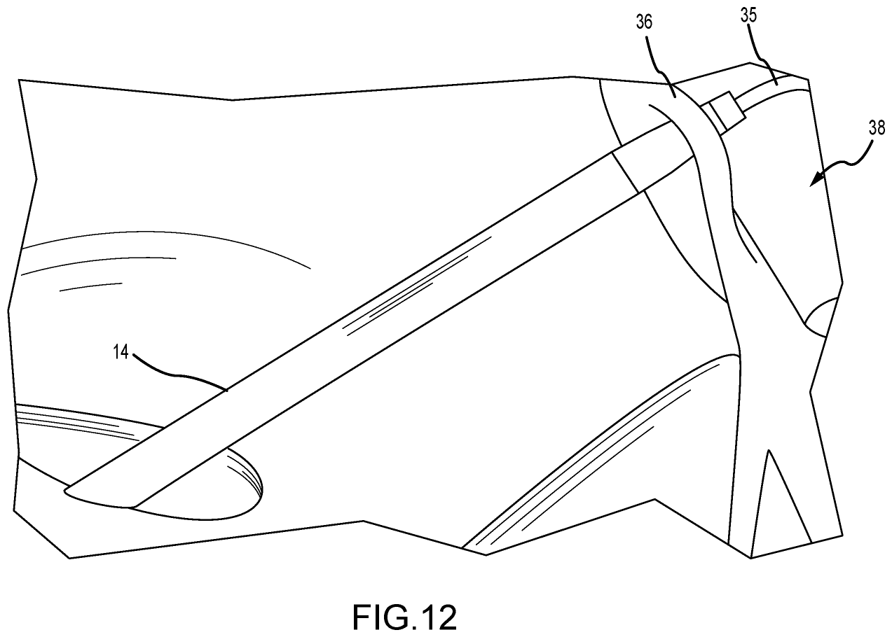

After dilator 14 (and, optionally, introducer 12) has crossed into left atrium 38, guidewire 35 can be advanced through lumen 24 and into left atrium 38, if desired, as shown in FIG. 12. Dilator 14 (and, optionally, guidewire 35) can then be withdrawn, and any desirable diagnostic or therapeutic device (e.g., an electrophysiology mapping catheter, an ablation catheter, or the like) can be advanced along guidewire 35 (if still present) and/or through introducer 12 into left atrium 38 according to methods that will be familiar to the ordinarily-skilled artisan. Advantageously, this simplifies transseptal crossing procedures by eliminating the need to exchange guidewires and needles through dilator 14 during the procedure.

Aspects of the disclosure also relate to adapters to secure dilator 14 to introducer 12. Those of ordinary skill in the art will appreciate that, in many extant devices, dilator 14 is secured to introducer 12 via a snap-fit locking mechanism. The ordinarily-skilled artisan will further appreciate, however, that these snap-fit locking mechanisms may be proprietary to particular manufacturers or suppliers. Moreover, introducers 12—including those from the same manufacturer or supplier—may have varying total lengths and/or usable lengths. These variations may be dictated, for example, by whether a particular introducer 12 is fixed-curve or steerable.

These realities limit a practitioner's ability to mix and match introducer 12 and dilator 14 to optimize the pairing for a particular procedure. The use of an adapter as disclosed herein can ameliorate these disadvantages by reducing the number of device models required to accommodate numerous combinations of introducers 12 and/or dilators 14 having different lengths, different curvatures, and other different configurations, including as between introducers 12 and/or dilators 14 from different manufacturers or suppliers.

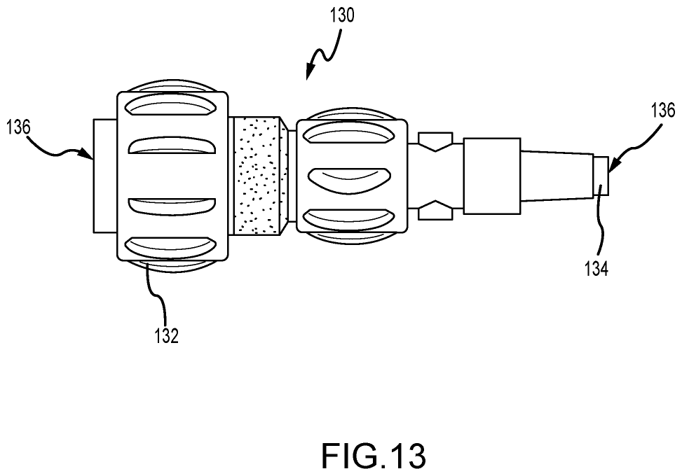

FIG. 13 depicts a representative adapter 130. As shown in FIG. 13, adapter 130 includes a proximal end 132 and a distal end 134. A lumen 136 to accommodate dilator 14 extends through adapter 130 from proximal end 132 to distal end 134.

Proximal end 132 of adapter 130 can include a fixation device to secure adapter 130 in place along the length of dilator 14 inserted therethrough. Suitable fixation devices include, without limitation, screws (e.g., set screws), clamps, valves (e.g., hemostasis valves) and the like.

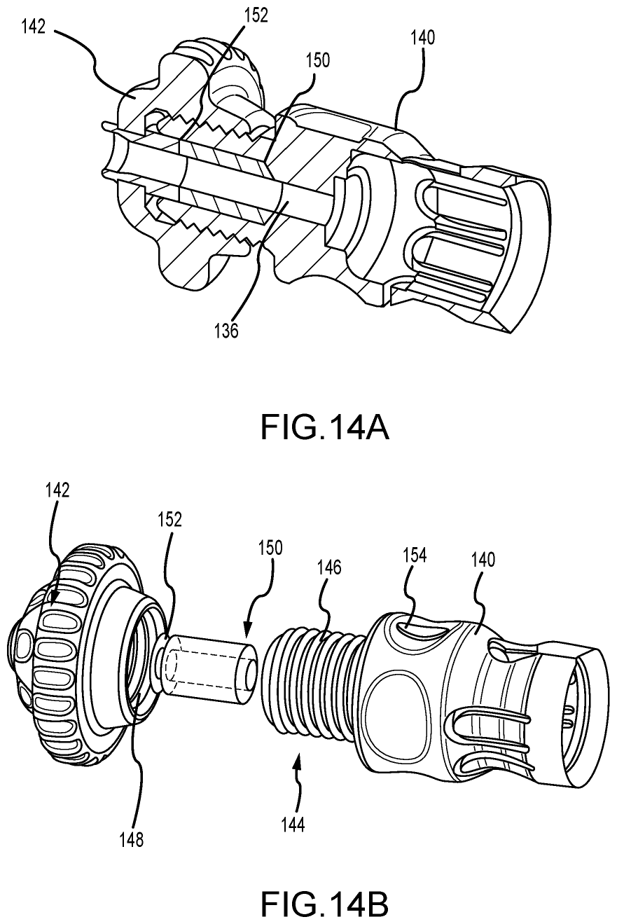

For instance, as illustrated to good advantage in the sectional and exploded views depicted in FIGS. 14A and 14B, respectively, adapter 130 can include a body 140 and a cap 142. The proximal portion 144 of body 140 can include external threads 146, while cap 142 can be internally threaded 148. As discussed further below, screwing cap 142 down onto body 140 with dilator 14 inserted through lumen 136 will secure adapter 130 at a desired position along the length of dilator 14.

Adapter 130 can also include an insert 150 within lumen 136. In some embodiments of the disclosure, insert 150 is made of silicone or another elastomer, which provides additional frictional resistance to adapter 130 sliding along the length of dilator 14. An optional washer 152 prevents cap 142 from impinging directly on insert 150.

Indicia 154 can be provided on body 140 of adapter 130 to indicate the orientation of dilator 14. For instance, where dilator 14 has a curvature at distal end 18, index 154 can be oriented to correspond to the inside of such curvature, thus signaling to a practitioner the rotational positioning of exposed portion 30b of energy delivery element 30.

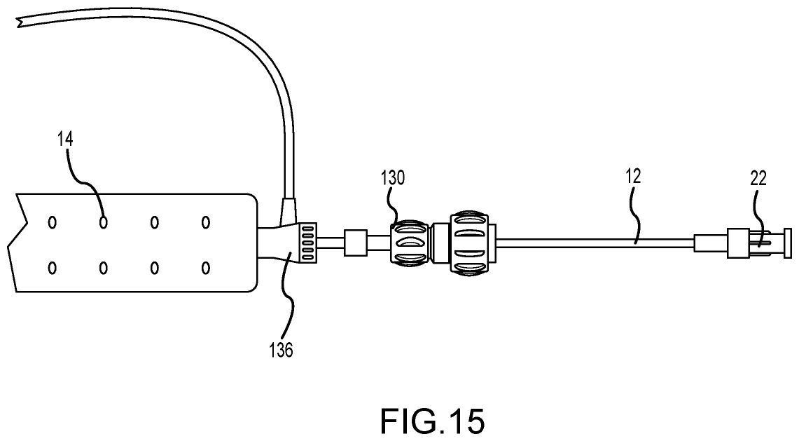

Distal end 134 of adapter 130 can include a snap-fit mechanism suitable for interconnection with a corresponding hub 136 (see FIG. 15) on the proximal end of introducer 12.

FIG. 15 depicts use of adapter 130 to secure dilator 14 to introducer 12, prior to advancing dilator 14 and introducer 12 into a patient's vasculature. In use, a practitioner will first insert dilator 14 through lumen 136 of adapter 130 and position adapter 130 near dilator hub 22 (e.g., at or near proximal end 16 of dilator 14).

Next, the practitioner will insert dilator 14 through introducer 12 such that distal end 18 of dilator 14 extends out of the distal end of introducer 12 by a desired amount. In embodiments where dilator 14 is plastically deformable (e.g., where body 20 includes a formable stainless steel hypotube 37), the practitioner can also shape dilator 14 into the desired shape either before or after insertion through introducer 12.

The practitioner can then slide adapter 130 distally along body 20 of dilator 14, towards introducer hub 136, eventually snapping distal end 134 of adapter 130 to introducer hub 136. This affixes adapter 130 to introducer 12.

To affix adapter 130 to dilator 14, the fixation device can be set in a locked position against dilator 14. For instance, where the fixation device is a hemostasis valve, it can be closed to seal against dilator 14. As another example, cap 142 can be tightened onto body 140 via mating threads 146, 148. Of course, by unlocking the fixation device (e.g., by re-opening the hemostasis valve or by unscrewing cap 142 from body 140), the practitioner can move dilator 14 relative to introducer 12 to adjust the extent to which distal end 18 of dilator 14 protrudes out of the distal end of introducer 12.

Although several embodiments have been described above with a certain degree of particularity, those skilled in the art could make numerous alterations to the disclosed embodiments without departing from the spirit or scope of this invention.

For example, it is contemplated that dilator 14 can incorporate a temperature sensor, such as a thermistor or thermocouple, proximate distal end 18.

As another example, rather than utilizing energy delivery element 30 attached to distal end 18 of dilator 14, the distal end of guidewire 35 can be made conductive, such as by removing any electrically-insulative coating from the exterior of guidewire 35 along a relatively short distal length on the order of at least about 0.015 inches.

As yet another example, rather than attaching energy delivery element 30 to distal end 18 of dilator 14, it can be attached to the distal end of introducer 12.

As a further example, lumen 24 can also be used to introduce irrigation fluid, contrast media, and the like into the body.

As a still further example, where main lumen 24 opens distally, dilator 14 can be used to introduce diagnostic and/or therapeutic devices into left atrium 38 without use of introducer 12.

As still another example, a transseptal catheterization according to the instant disclosure can utilize what is known as “rail delivery,” where guidewire 35 is generally advanced along the outside of dilator 14, enters dilator 14 through side port 26b, and exits dilator 14 distally through distally-facing opening 26a.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An apparatus for transseptal catheterization, comprising:
a dilator having a proximal end, a distal end, and a lumen extending between the proximal end and the distal end; and
an energy delivery element attached to the distal end of the dilator and configured to deliver sufficient energy to a tissue adjacent the distal end of the dilator to permit the dilator to penetrate the tissue.

2. The apparatus according to claim 1, wherein the energy delivery element comprises an electrode.

3. The apparatus according to claim 1, wherein the energy delivery element comprises a hollow cylindrical element inserted into the lumen at the distal end of the dilator.

4. The apparatus according to claim 3, wherein an interior-facing energy-emitting surface of the energy delivery element is covered with an energy-inhibiting material, and wherein at least a portion of a distal-facing energy-emitting surface of the energy delivery element is exposed.

5. The apparatus according to claim 4, wherein between one-quarter and three-quarters of the distal-facing energy-emitting surface of the energy delivery element is exposed.

6. The apparatus according to claim 1, further comprising a guidewire configured to be inserted through the lumen of the dilator.

7. The apparatus according to claim 6, wherein the lumen of the dilator further comprises a central lumen terminating at an opening in the distal end of the dilator and a side lumen terminating at a side port through a wall of the dilator proximate the distal end of the dilator, and wherein the guidewire is configured to be inserted through the side lumen.

8. The apparatus according to claim 1, further comprising a magnetic localization element carried by the dilator proximate the distal end.

9. The apparatus according to claim 1, further comprising an introducer sheath having a distal end, a proximal end, and a lumen extending therebetween, wherein the dilator is configured to be inserted through the lumen of the introducer sheath.

10. The apparatus according to claim 1, wherein the dilator further comprises a hypotube, wherein the hypotube defines the lumen, and wherein the hypotube further comprises at least one of:
a plurality of slots cut into a wall of the hypotube, wherein each slot of the plurality of slots extends at least partially around a perimeter of the hypotube and least partially through the wall of the hypotube; and
a coil.

11. The apparatus according to claim 1, wherein the lumen comprises:
a main lumen; and
at least one secondary lumen.

12. The apparatus according to claim 11, wherein the at least one secondary lumen comprises a conductor lumen configured to accommodate an electrical conductor that is conductively coupled to the energy delivery element.

13. The apparatus according to claim 11, wherein the at least one secondary lumen comprises a pressure-measuring lumen having an open distal end.

14. An apparatus for transseptal catheterization, comprising:
a dilator having a proximal end, a distal end, and a lumen extending therebetween; and
a guidewire configured to be inserted through the lumen of the dilator and having a distal end,
wherein at least one of the distal end of the dilator and the distal end of the guidewire comprises an energy delivery element configured to deliver sufficient energy to an adjacent tissue to permit penetration of the adjacent tissue.

15. The apparatus according to claim 14, wherein the energy delivery element comprises an electrode attached to the distal end of the dilator.

16. The apparatus according to claim 14, wherein the energy delivery element comprises an exposed conductive distal end of the guidewire.

17. The apparatus according to claim 14, further comprising an introducer sheath having a distal end, a proximal end, and a lumen extending therebetween, wherein the dilator is configured to be inserted through the lumen of the introducer sheath.

18. An apparatus for transseptal catheterization, comprising:
an introducer including an introducer hub attached to a proximal end of the introducer;
a dilator including an elongate body and an energy delivery element attached to a distal end of the elongate body, wherein the energy delivery element is configured to deliver sufficient energy to a tissue adjacent the distal end of the dilator to permit the dilator to penetrate the tissue; and
an adapter having a distal end, a proximal end, and a lumen extending between the distal end of the adapter and the proximal end of the adapter, wherein the lumen is configured to receive the elongate body of the dilator therethrough, the adapter further comprising:
a connector at the distal end of the adapter, wherein the connector is configured to releasably secure the adapter to the introducer hub; and
a fixation device at the proximal end of the adapter, wherein the fixation device is configured to releasably secure the adapter to the dilator when the elongate body of the dilator is received within the lumen of the adapter.

19. The apparatus according to claim 18, wherein the fixation device comprises one or more of a screw, a valve, and a clamp.

20. The apparatus according to claim 19, wherein the fixation device comprises a hemostasis valve.
